(19) United States
(12) Patent Application Publication
(10) Pub. No.: US 2009/0048565 A1
(43) Pub. Date: Feb. 19, 2009

(54) Device For Displaying Data Relevant For a Diabetic Patient

(76) Inventor: John Thrane Hansen, Vipperod (DK)

Correspondence Address: NOVO NORDISK, INC.;INTELLECTUAL PROPERTY DEPARTMENT, 100 COLLEGE ROAD WEST, PRINCETON, NJ, 08540, US

(21) Appl. No.: 11/792,645

(22) PCT Filed: Dec. 7, 2005
(86) PCT No.: PCT/EP05/56554

§ 371 Date: Jun. 2, 2008

(30) Foreign Application Priority Data
Dec. 8, 2004 (DK) PA 2004 01905

(51) Int. Cl. A61M 5/31

(52) U.S. Cl. 604/189

(57) ABSTRACT

This invention relates to a device with a display (8) for displaying graphics, text and/or combinations thereof, the device has a processor (230) that is interfaced with said display, where the processor can compute and cause the display to display when to administer bolus insulin in which dose(s), when insulin at several occasions in fact were administered and in which doses at these occasions. Moreover, the processor can compute data linking a treatment regimen and insulin administered in the past. A display of the relations between the treatment regimen and the insulin administered in the past are possible. Moreover, a display of data, e.g. a graph, showing insulin administered in the past for longer periods, e.g. more days, months, etc. is possible. The data is useful in a dialogue between a health care person and a diabetic patient.

### FIELD OF THE INVENTION

The present invention relates to the field of health management and in particular, self-medication and treatment. More particularly the invention relates to a device capable of displaying data relevant for a diabetic patient.

### BACKGROUND OF THE INVENTION

Health problems in humans can broadly be clubbed under two categories i.e. acute and chronic. Acute diseases are sudden problems in the body that have a well-defined method for treatment and once treated; the patient is back to his normal life.

Chronic diseases on the other hand are problems that are faced by a person because of some metabolic dysfunctions. These kinds of problems are difficult to treat and require a kind of control. This control apart from regular medication and other health care regime also requires a life style management from the patient.

Diabetes is one such kind of chronic disease that requires continuing medical care and patient self-management education so as to avoid complications. Diabetes is also classified as a chronic disease. Lack of insulin (produced by pancreas) in the body results in a rise in the blood sugar level, which in turn has various effects such as excessive thirst, frequent urination, weakness and excess of ketones in the bloodstream.

Two main kinds of insulin used in diabetes treatment are bolus insulin and background insulin, the latter is also referred to as basal insulin. Bolus insulin supplies a burst of insulin and is usually administered before or in relation to a meal. The two types of bolus insulin are rapidacting and short-acting. Rapid-acting bolus insulin works quickly and leaves the body quickly. Short-acting bolus insulin stays in the body longer.

Basal insulin or background insulin supplies a low level of insulin throughout the day and overnight. The three kinds of background insulin are intermediate-acting, prolonged intermediate-acting and long-acting. Of the three different background insulin, long-acting insulin stays in the body the longest.

In order to keep the blood sugar level in check, diabetics administer doses of insulin at regular intervals of time. However this is not a cure but just a part of the treatment. A diabetic Health Management program would typically involve other elements such as regular exercise, food intake monitoring etc. A balance between the food intake and exercising etc has to be maintained so as to make the body behave as close as possible to a normal body. U.S. Pat. No. 6,248,090B1 discloses a syringe having a dose setting mechanism, a button which can be operated to inject a set dose, a switch operated at a time between the start and completion of injection, and an electronic presentation of parameters such as the size of a set dose and the size of the last dose administered. The syringe also has a stop watch which is reset and started responsive to operation of the switch. The electronic presentation includes an indication of the number of hours elapsed from the activation of the switch, and may also include, for a predetermined period initially following the activation of the switch, a presentation of the number of seconds elapsed. The latter presentation can provide a visual indication to the patient of the length of time, after the injection button has been actuated to inject the dose, that the needle should remain inserted in the skin.

Drug administration devices become more sophisticated and more affordable; it is possible for patients to self-treat certain conditions. Diabetes for example, is a medical condition that often requires a patient to monitor her own blood glucose level and then make an adjustment to self-administered insulin doses. Likewise, hemophilia and other chronic long-term illness often are best managed when the patients monitors their condition and make adjustments to their treatment accordingly. While one-on-one care with a physician is often critical, the patient is often in the best position to make small and short term adjustments to their own treatment. Advances in computers and electronics have made it possible to monitor more and more medical conditions. Unfortunately, the people who tend to have the long-term illness that are best suited for self-treatment tend to be older, have poorer eyesight, and less comfort with electronic devices. According, medical monitors for self-treatment need to provide accurate information in a simple and intuitive manner that is easy to understand and provides the patient viewing the information with sufficient data to adjust their treatment.

The breakthrough in disease self-management especially in diabetes came with the introduction of portable self-operated drug administration devices. These devices are not only easy to use but also safe. For example devices to inject insulin (for diabetes patients), inhalers (for asthma patients or diabetes patients as well), blood sample collection device, e.g. blood glucose meter, etc are widely available in the market. These devices have the dual purpose of administering the drug dosage to the patient as well as they can have advanced functionality inbuilt such as recording of patient's data to establish diary data and setting reminders for him. The device can have an alarm system as well as a display means for analyzing of the recorded data or they can transfer the data through some communication channel to an external computing device with better processing capabilities and/or bigger display means. International Publication Numbers WO 00/32088, WO 03/005891 and WO 03/015838 all describe such medical devices, networks and method of their operation along with some of the possibilities in the domain. These publications are incorporated herein in entity by way of reference.

In future more and more diabetics will have a raising need for more daily insulin injections, when they are chancing over from Insulatard®/Actrapid® treatment to using insulin analogs as ex Levemir® and NovoRapid®-analogs which allows a more flexible lifestyle for example more varying and heavy physical activities, displacing mealtimes and leaving snacks.

Diabetics, who want this flexible lifestyle, simply will have a latent need for more and IU-varying daily insulin injections—typically between 5 to 10 injections round the clock, e.g. diabetics which are using as an example between 5 to 10 IU-varying insulin injections round the clock in a multiple basal/bolus treatment, and are using insulin analogs such as example Levemir® and NovoRapid®.

It can be hard for the diabetic to remember how much insulin that was administered or was to be administered for the whole day round the clock. This can have two consequences, either the patient administers more insulin than prescribed, e.g. since he forgot what he took and therefore—to compensate—administers another additional dose; or the patient administers less insulin than prescribed, e.g. since he thought he did administer a dose and he therefore will not administer the—in fact missing—dose. In any of the two cases, the patient is not following his treatment regimen.

The display of data as offered by mentioned devices/software have the drawback that the user cannot see—e.g. from a treatment regimen for the diabetic patient—when to administer insulin in which dose(s), further the user cannot see on his device when insulin at several occasions in fact were administered and in which doses at these occasions. Moreover, computations linking data from the treatment regimen to insulin in fact administered are not performed, thus no subsequent display of the relation between the treatment regimen and the insulin administered in the past is available. Moreover, a display of a graph showing insulin administered in the past for longer periods, e.g. more days, months, etc. is not available.

The present invention overcomes these drawbacks since said device in general comprises

a display for displaying graphics, text and/or combinations thereof,
a processor (230) that is interfaced with said display.

In an embodiment of the invention, the device for informing a diabetic patient about dose of insulin administered, said device has the processor configured to:
be provided with data comprising logged doses of bolus insulin administrations in a registration period, and cause the display to display two or more of said provided logged doses.

Said logged doses of bolus insulin administrations can be received from an external device, alternatively these data are keyed in or dialed on said device by the user, e.g. the diabetic patient or a health care person.
The processor is optionally configured to cause the display to display two or more corresponding point of times of administrations.

It is thus an advantage that the diabetic patient does not need manually to enter doses of insulin administered in order to maintain a manual logbook. Instead the displayed data will help the patient check whether insulin was administered or not, in which dose and at which point of times. Accordingly, the patient most like will only administer doses of insulin as intended, thus avoiding administering too much or too little, consequently avoiding a hypoglycaemic and a hyperglycaemic reaction, respectively, and the effects of the reactions. Furthermore, typically the patient will know which total amounts, e.g. targets, which normally are administered during a period, with that information in mind the patient can—when comparing with shown data—choose to administer more or less, or postpone or hasten a medication to reach said targets.

Furthermore, he can use the data to have a dialogue with a health care person discussing the reasons why, e.g. too much or too little doses of insulin were administered during the registration period, e.g. during the day.

By means of the displayed data, the diabetic patient then has a tool to counter react, e.g. in the case where generally too much insulin administered, he could then choose to lower his food consumption, and accordingly less insulin is needed, which finally in the long run could avoid weight gain.

When the dose of insulin administered—as currently displayed for the registration period, e.g. a day—equals the dose of insulin to be administered at the same point of time the diabetic patient then is in compliance with his treatment regimen on that point of time.

In an embodiment of the invention, the device for informing a diabetic patient about dose of insulin administered, said device has the processor configured to:
be provided with data comprising logged doses of bolus insulin administrations in a registration period, and
calculate a number of current sums of said provided logged doses of bolus insulin administrations, and
cause the display to display two or more of said number of current sums of said provided logged doses of bolus insulin administrations.

The processor is optionally configured to cause the display to display two or more corresponding points of times of administration.

It is thus an advantage for the diabetic patient, e.g. if the sum of doses of insulin administered—as displayed for the registration period, e.g. a day—equals the sum of doses of insulin to be administered the diabetic patient, then he knows that he, on that day, is in compliance with his treatment regimen. Conversely, in the case that said sum differs from the sum of doses of insulin to be administered, he is not in compliance with his treatment regimen.

In an embodiment of the invention, the device for informing a diabetic patient about dose of insulin to be administered in a treatment regimen, said device has the processor configured to be provided with data comprising doses of bolus insulin administration prescribed for the diabetic patient in a registration period, and cause the display to display one or more of said doses.
The processor is optionally configured to cause the display to display one or more points of times of prescribed administration.

In an embodiment of the invention, the device for informing a diabetic patient about dose of insulin to be administered in a treatment regimen, said device has the processor configured to:
be provided with data comprising doses of bolus insulin administration prescribed for the diabetic patient in a registration period,
calculate a number of current sums of doses of bolus insulin from said treatment regimen in said registration period, and
cause the display to display one or more of said number of current sums.
The processor is optionally configured to cause the display to display one or more points of times of prescribed administration relating to said number of current sums.

The patient could choose to use the data—from the two embodiments—to have a dialogue with a health care person discussing the reasons why he is to administer which doses of insulin at certain points of times to reach the dose or the summed doses during the registration period, e.g. during the day. Consequently, the health care person could advice him what to do to be in compliance with his treatment regimen. The health care person could, in his advice, give the patient the option of postponing or hastening administration while still reaching the target or summed target at the end of the registration period, e.g. at the end of the day.

In an embodiment of the invention, the device for informing a diabetic patient about doses of insulin administered in relation to the treatment regimen for the diabetic patient, said device has the processor configured to:
1) be provided with data comprising logged doses of bolus insulin administrations in a registration period,
2) be provided with data comprising doses of bolus insulin administrations prescribed for the diabetic patient in said registration period,
calculate a first number of current sums from 1) said provided logged doses of bolus insulin administrations in said registration period,
calculate a second number of current sums from 2) doses of bolus insulin administrations prescribed for the diabetic patient in said registration period,
calculate the number of differences between said first and second number of current sums, where any positive difference indicates more bolus insulin administered than prescribed at the point of time of administration in the registration period, and where any negative difference indicates less bolus insulin administered than prescribed at the point of time of administration in the registration period, and
cause the display to display one or more of said differences.
The processor is optionally configured to cause the display to display one or more corresponding points of times of administration.

The displayed data, i.e. data showing differences between current sums of bolus insulin in fact administered and bolus insulin prescribed at points of times will help the patient check whether insulin was administered or not, in which dose compared to the prescribed dose and at which point of times. A positive difference indicates more insulin administered than prescribed, conversely a negative difference indicates less insulin administered than prescribed at the point of time of administration in the registration period. Furthermore, said differences show at each point of time whether the diabetic patient, e.g. during a day as the registration period, have administered more or less bolus insulin seen in relation to the prescribed doses, typically said differences are shown three to four times a day (i.e. said points of times), when the day has been selected as the registration period.

By means of the displayed data, i.e. the difference over a registration period, e.g. a day, the diabetic patient then has a tool to be in compliance with his treatment regimen, e.g. trying to get the difference to be close to zero IU. When the treatment regimen is strictly followed, he should then avoid any weight gain.

Furthermore, the patient could choose to use the data, i.e. the difference, to have a dialogue with a health care person discussing the reasons why—at which points of times—he actually did administer more or less doses of insulin as compared to his treatment regimen during the registration period, e.g. during the day. Consequently, the health care person could advice him what to do and when to be in compliance with his treatment regimen. The health care person could in his advice give the patient freedom to postpone or hasten administration in order to reach an almost zero difference at the end of the registration period, when it is reached, typically at the end of the day. A zero difference at the end of the registration period implies that the he is in compliance with his treatment regimen.

In an embodiment of the invention, the device for informing a diabetic patient about doses of insulin administered in a period of time passed, said device has the processor configured to:

from said period of time passed, which is two or more registration periods of equal length,
compute a sum of logged doses of bolus insulin administered for each registration period, and
cause the display to display said sum for two or more corresponding registration periods.

The processor is optionally configured to cause the display to display data, such as date, day number, week number, quarter, year identifying each of said registration periods.

It is thus an advantage that the displayed data on the device could assist the patient to see e.g. for said period of time passed—whether insulin administered in these days follow an increasing or a decreasing trend, or whether there is no trend, but a status quo, i.e. the diabetic patient in the latter case administers the same total amount of doses every day. Furthermore, he can use the data to have a dialogue with a health care person discussing the reason(s) why, e.g. an increasing trend is the case. The increasing trend, i.e. more and more insulin were administered each day, which most likely is caused by more ingestions of food, and consequently very likely could cause an increasing weight for the diabetic patient, which finally could cause obesity problems.

In an embodiment of the invention, the device for informing a diabetic patient about doses of insulin administered in relation to a treatment regimen for the diabetic patient in a period of time passed, said device has the processor configured to:

from said period of time passed, which is two or more registration periods of equal length, calculate a number of differences between:
a computed sum of logged doses of bolus insulin administered for each registration period,
a computed sum of prescribed doses of bolus insulin to be administered for each registration period,
wherein the processor is further configured to:
cause the display to display two or more number of differences for corresponding registration periods, where a positive difference indicates more bolus insulin administered than prescribed for the corresponding registration period, and where a negative difference indicates less bolus insulin administered than prescribed for the corresponding registration period.

The processor is optionally configured to cause the display to display data, such as date, day number, week number, quarter, year identifying each of said registration periods.

It is thus an advantage, that the displayed data, i.e. said differences as displayed on the device indicate to the diabetic patient which day(s) from the time passed, i.e. for two or more registration periods, where the summed dose of insulin in fact administered was equal to prescribed summed dose of insulin, when, i.e. which day(s) where less bolus insulin was administered as compared to prescribed doses, and in which day(s) more bolus insulin was administered than the prescribed dose. A positive difference is meant to indicate more insulin administered than prescribed, and, conversely a negative difference indicates less insulin administered than prescribed.

For the days, where more bolus insulin was administered than prescribed, this is most likely caused by more ingestions of food in these days as compared to the other days in the time passed, and consequently, excessive ingestion of food very likely could cause an increasing weight for the diabetic patient, especially if this situation persists. Further more, there can be displayed data on the device, such as a date, a day number, a week number and a quarter, which, when related to the positive difference, could indicate these registration periods where more insulin was administered than prescribed. Correspondingly data, which, when related to the negative difference, could indicate other registration period(s), e.g. which period displayed as dates of days, where less bolus insulin was administered than prescribed.

In an embodiment of the invention, the device for informing a diabetic patient about doses of insulin administered in relation to the treatment regimen for the diabetic patient in a period of time passed, said device has the processor configured to:

from said period of time passed, which is two or more registration periods of equal length, calculate a number of differences between:
a computed sum of logged doses of bolus insulin administered for each registration period,
a computed sum of prescribed doses of bolus insulin to be administered for each registration period,
wherein the processor is further configured to:
calculate a number of currents sums based on said number of differences for a corresponding number of registration periods, and
cause the display to display one or more of said number of currents sums for corresponding registration periods, whereby any positive displayed current sum indicates more bolus insulin administered than prescribed, and where any negative displayed current sum indicates less bolus insulin administered than prescribed.

The processor is optionally configured to cause the display to display data, such as date, day number, week number, quarter, year identifying each of said registration periods.

It is thus an advantage, that the displayed data on the device, i.e. said sums of differences between, etc—a positive displayed current sum indicates more bolus insulin administered than prescribed for the entire period passed, and a negative displayed current sum indicates less bolus insulin administered than prescribed also for the entire period passed.

If said “sums of differences between”, etc is zero—as displayed for the registration periods, e.g. for two or more days—then the diabetic patient was in general in compliance with his treatment regimen seen over these two days.

However, a day with 2 IUs less administered as prescribed in the case above could be counter-balanced with another day with 2 IUs more administered as prescribed, since in this case—when considered over these two day—sums of differences gets zero (2 IU-2 IU), and thus the diabetic patient was in compliance with his treatment regimen, in general, but seen over these two days.

In general, the patient could choose to use the data to have a dialogue with a health care person discussing the reasons why, e.g. too much or too little doses of insulin were administered during a day or during more registration periods, e.g. during more days. Consequently, the health care person could advice him what to do to be in compliance with his treatment regimen.

According to the invention the diabetic patient is assisted in remembering how much insulin that was administered for and during the whole day round the clock, and for days passed as well. Thereby, the patient has a much higher probability to take the amount of insulin as prescribed, thus the patient will to a much higher certainty follow a prescribed treatment.

IU, International Units of insulin's can be summed. If the number of IUs administered for bolus insulin is 5 at one point of time and if the number of IUs administered for bolus insulin at another point of time, the resulting—i.e. the sum—of IUs administered of bolus insulin then is 12 for the period between these points of times.

As discussed, the invention may be carried out on a device, e.g. a drug administration device, it may be a doser for injection of insulin in various concentrations, it may be in a simpler form as an electronic syringe equipped with display capabilities. For example U.S. Pat. No. 6,540,672, U.S. Pat. No. 6,656,114, US2002010432 and US2003032868 all disclose intelligent drug administration devices, which are hereby incorporated by reference in its entirety.

The invention may as well be carried on a drug administration device in form of a pump also capable of infusing insulin in various concentrations as general known in the art. Alternatively, the drug administration device may be an inhalation device: various inhalation devices exist that aid in depositing a liquid aerosol or dry aerosol powder into a patient's lungs. For example, U.S. Pat. No. 5,888,477 (which is hereby incorporated by reference in its entirety) discloses an inhaler with robust features that may be used for insulin delivery. U.S. Pat. No. 5,785,049 to Smith et al. (which is hereby incorporated by reference in its entirety) discloses a device suitable for powdered medication delivery.

Thus, in the present context, the term ‘drug administration device’ is taken to mean, an injector type device (such as a pen injector or a jet injector) for delivering a discrete dose of a liquid medication (possibly in the form of small drops), a medication pump for continuous delivery of a liquid medication, an inhaler, spray or the like for delivering a discrete or continuous dose of a medication in vaporized, ‘atomized’ or pulverized form. The invention may as well be implemented on an electronic device, such as a personal digital assistant, a cellular phone or on a blood glucose meter.

The invention will be explained more fully below in connection with preferred embodiments and with reference to the drawings, in which:

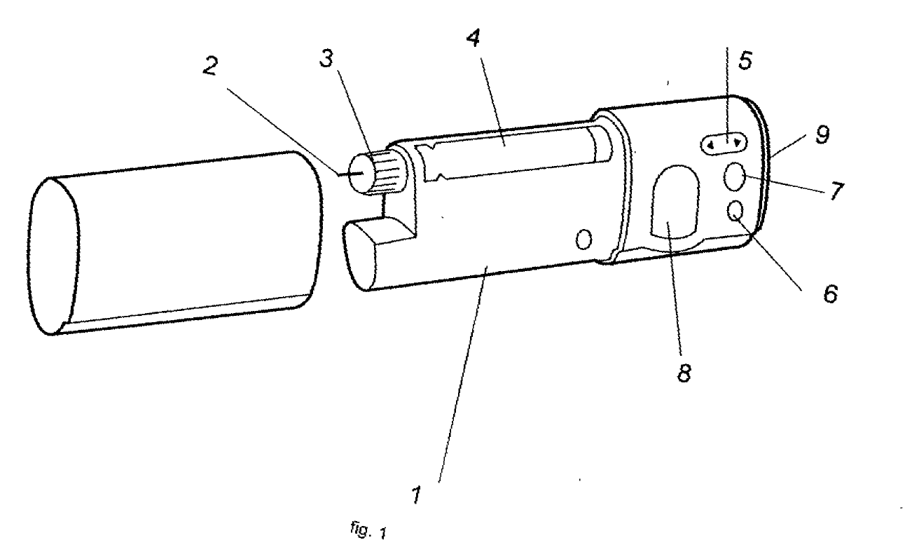

FIG. 1 shows an exemplary embodiment of a device,

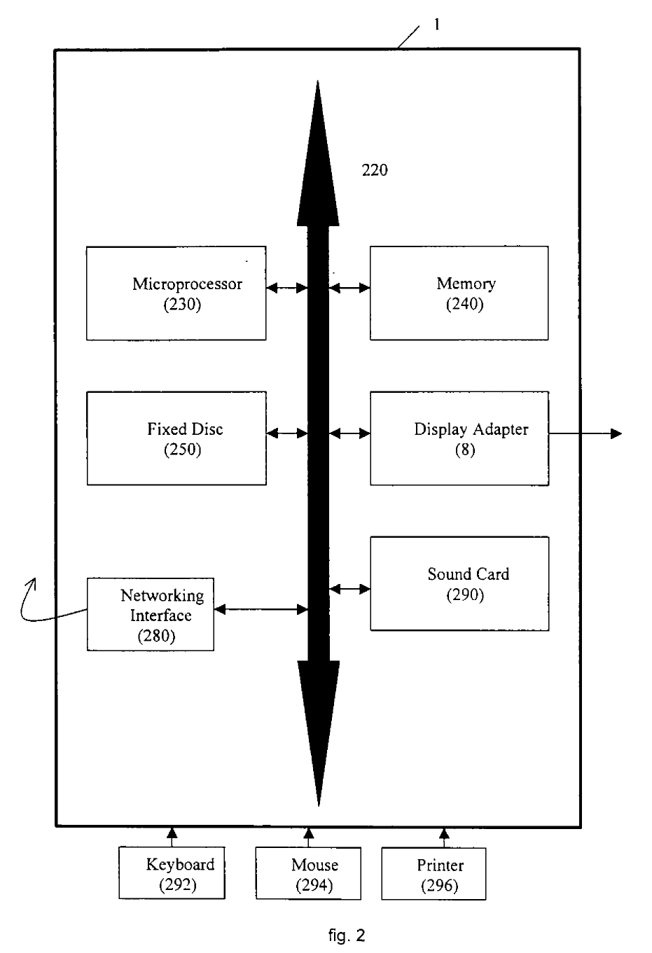

FIG. 2 shows an exemplary embodiment of the devices' electronic circuit.

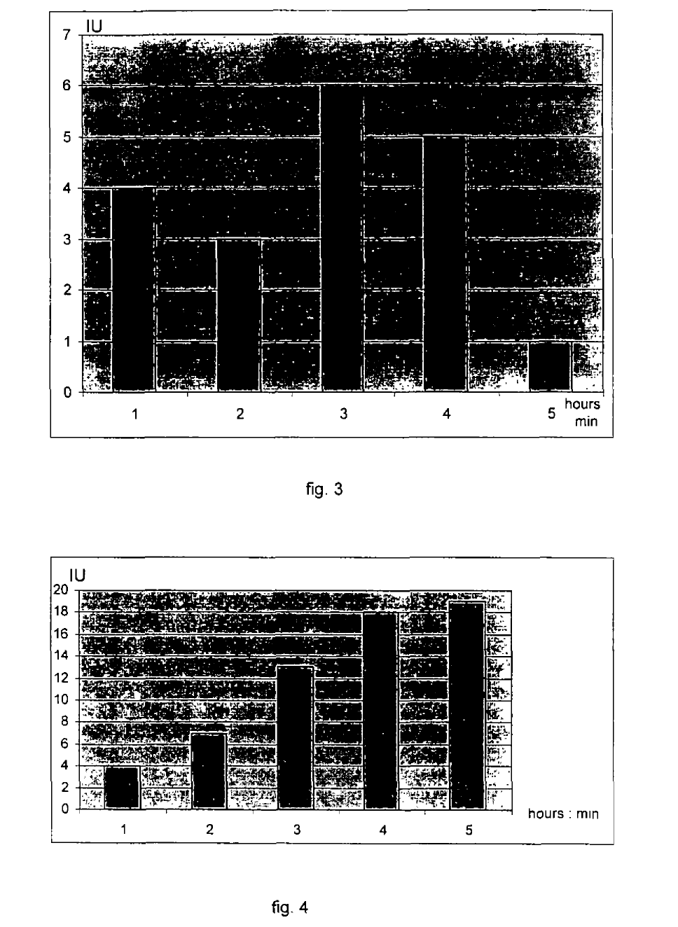

FIG. 3 shows a display showing provided logged doses of insulin administered,

FIG. 4 shows a display showing provided currently summed logged doses of insulin administered,

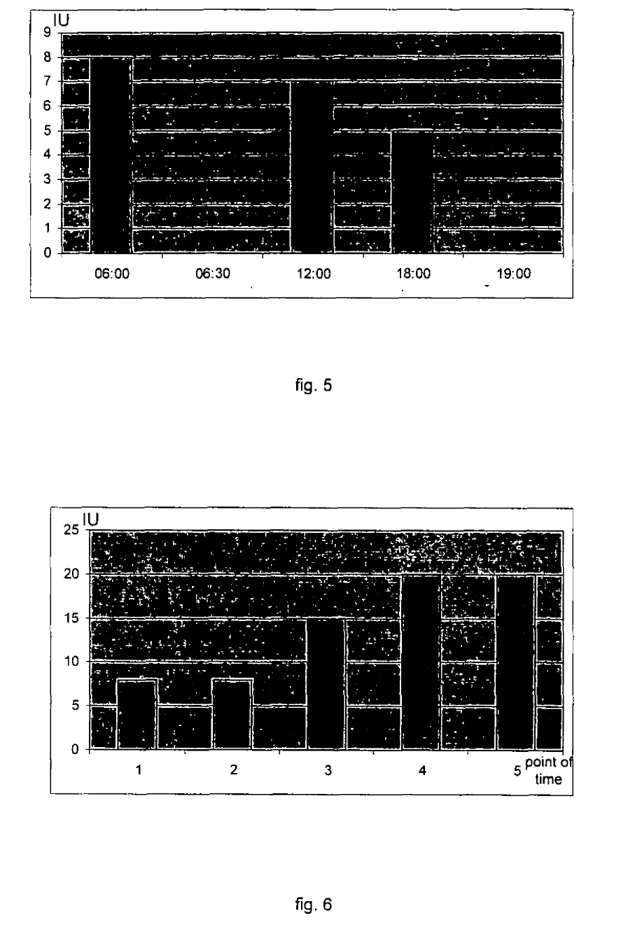

FIG. 5 shows a display showing dose of insulin to be administered in a treatment regimen,

FIG. 6 shows a display showing currently summed dose of insulin to be administered in a treatment regimen,

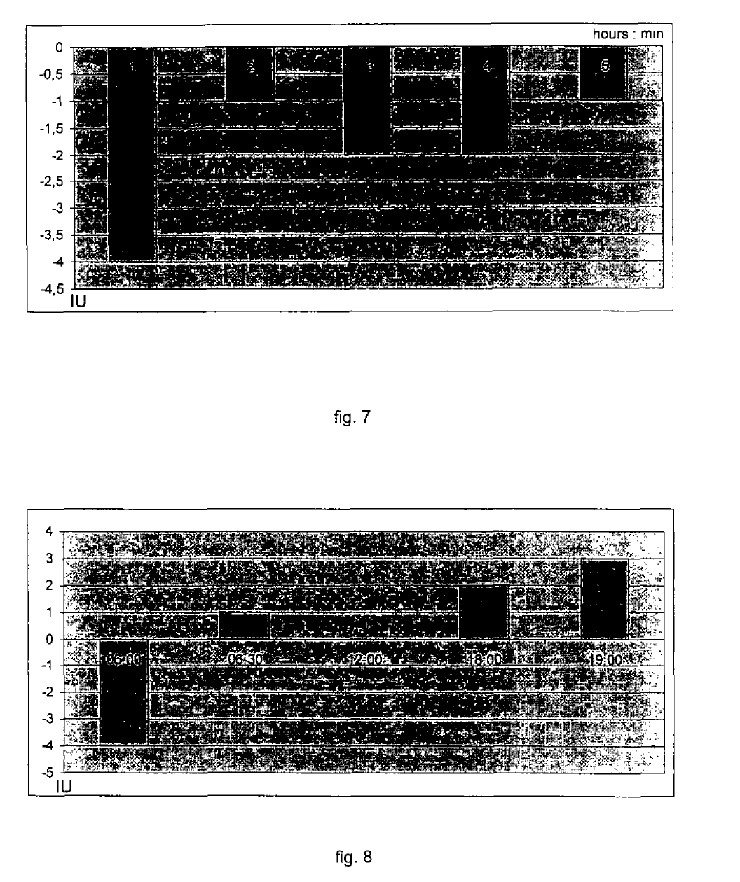

FIGS. 7 and 8 show a display showing differences between insulin administered and prescribed at points of times during a registration period,

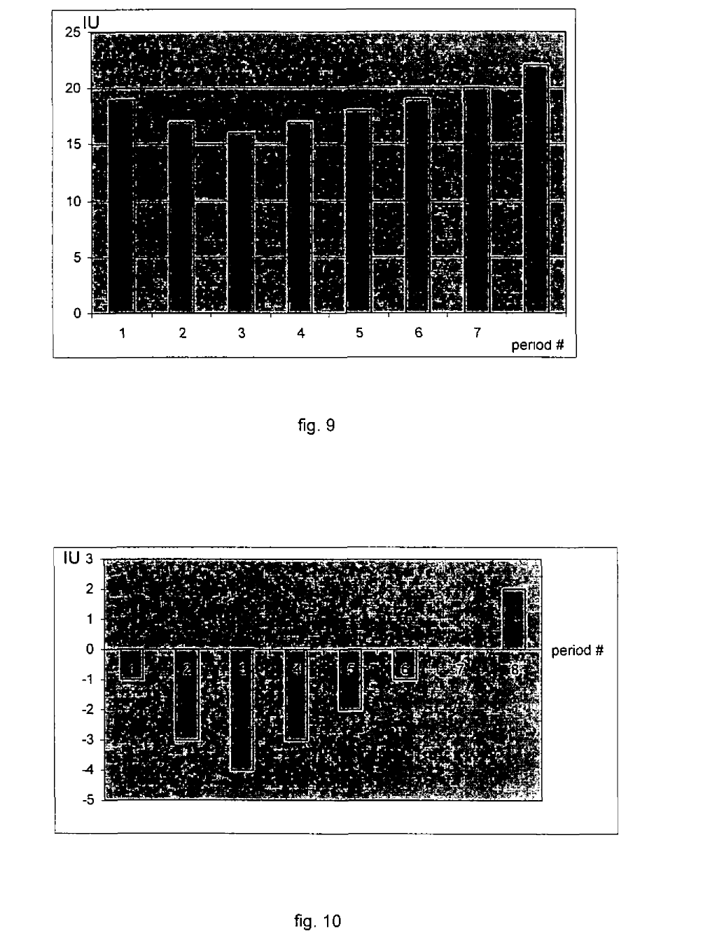

FIG. 9 shows a display showing sums of insulin administered during eight registration periods,

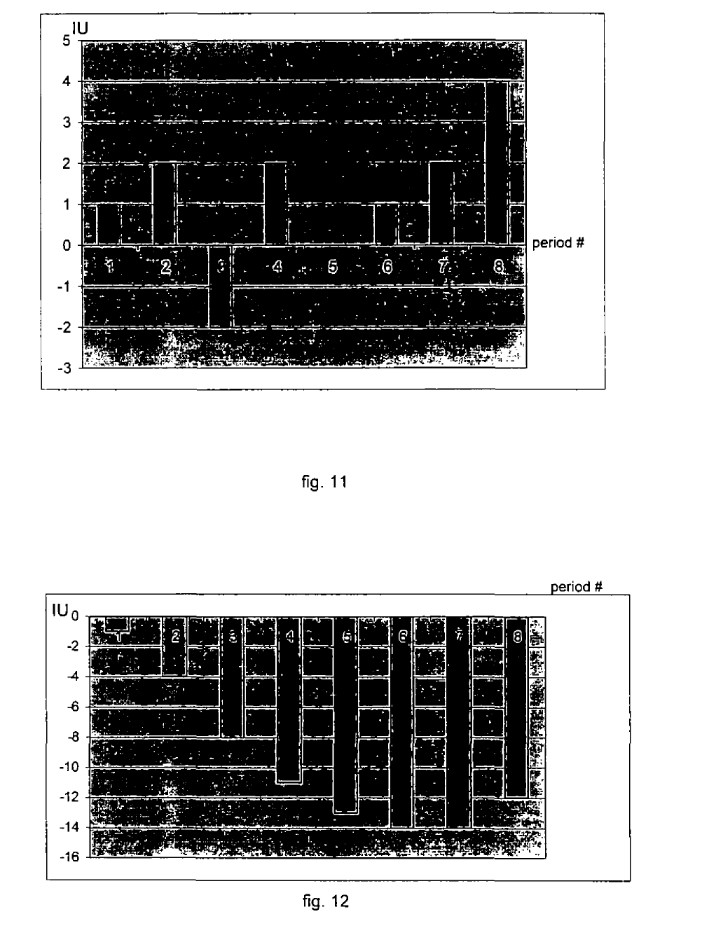

FIGS. 10 and 11 show a display showing differences between insulin administered and prescribed from eight registration periods, and

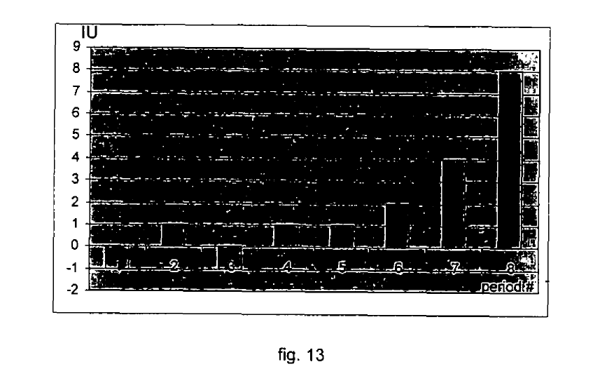

FIGS. 12 and 13 show a display showing the summed differences from FIGS. 10 and 11 for 8 registration periods.

Throughout the drawings, the same reference numerals indicate similar or corresponding features, functions, etc.

FIG. 1 discloses an exemplary embodiment of a device 1, e.g. a drug administration device having housing. An injection needle 2 is connected to a needle assembly 3 connected to the distal end of the housing and communicates with a reservoir 4, e.g. a cartridge or ampoule containing the medicine to be administered, e.g. an injection of basal or bolus insulin.

A plurality of operating buttons 5, 6, 7, 9 in an exemplary embodiment of the drug administration device is provided, these comprise a dose setting button 5 for setting a dose to be injected, an accept button 6 for accepting the dialled dose, an escape button 7 for moving backwards in the menu and an injection button 9.

In order to perform an injection the user could dial the size of the dose to be injected using the dial up/dial down button 5. As the dose is dialled, the size of the dose is displayed in the display 8. When the set dose is dialled to an adequate size, the user operates the accept button 7 thereby confirming the set dose. After having inserted the injection needle 2 into a tissue of a diabetic patient, the user operates the injection button 9 to release the set dose.

Said display may further be used to display what is shown in FIGS. 3 to 13 corresponding to what are shown in tables 1 to 5 in a graphical form, in textual form or in a combination of both forms.

FIG. 2 discloses an exemplary embodiment of the devices' electronic circuit. Said device can be a drug administration device or a blood glucose meter as well.
This display of data can be implemented in a method which can be run on any general purpose device/computer system as shown in the figure, which shows its internal structure. The computer system (210), e.g. a device consists of various subsystems interconnected with the help of a system bus (220). The microprocessor (230) communicates and controls the functioning of other subsystems. Memory (240) helps the microprocessor in its functioning by storing instructions and data, e.g. such as medication of bolus insulin performed to be performed at which points of times from one or more registration periods. Fixed Drive (250) may be used to hold these data, e.g. in a database structure and instructions permanent in nature like the operating system and other programs, furthermore the fixed drive may contain data for a subsequent display. Display adapter (260) is used as an interface between the system bus and the display device (8), which is generally a monitor or a display. In other words, the display is interfaced with said processor, where the processor can be configured to cause the display to display various data as graphics, numbers text and any combinations thereof. This monitor or display can be used to display various data, such as medication of bolus insulin performed, to be performed from a treatment regimen at various point of time. Furthermore, sums of said data and other manipulations of said data can be shown of the display, as numbers, text, graphics, e.g. bar graph, pie chart, etc, the user of the device may determine what to show and how. The network interface (280) may be used to connect the computer with other computers on a network through wired or wireless means. These devices on the network can also be drug administration devices. These drug administration devices can be capable of storing patient related data such as drug dosage, point of times for drug dosage, e.g. for bolus insulin. These devices communicate with the computing device using various communication mediums. The communication means can be wired or wireless such as cable, RS232, Bluetooth, infrared etc using various communication protocols such as TCP/IP, SSL etc. The computer system might also contain a sound card (290). The system may be connected to various input devices like keyboard (292) and mouse (294) and output devices like printer (296). Various configurations of these subsystems are possible. It should also be noted that a device or system implementing the present invention might use less or more number of the subsystems than described above.

This arrangement between the device and the computing system—on both of which the invention can reside—can be as simple from a one to one link between the two. But at the same time it can also be expanded and customized as per the need to establish an efficient patient-doctor-relative-peer network. For example the computing system may periodically logon to a Local Area Network, or Internet to transmit the user readings, e.g. what doses of bolus insulin was administered at which point of times on a remote database server that might be used to generate reports or receive a treatment regimen for the diabetic patient from a different computing system such as that of a doctor, relative of the patient and the like. These computing devices can be general-purpose desktops or other variations such as laptop, cell phones, PDAs, blood glucose meters, etc.

The method is incorporated in the aforementioned computing devices as by instructions in the software that are carried out by the computer system. Again, the software may be implemented as one or more modules for implementing the method.

In particular, the software may be stored in a computer readable medium, including the storage device or that is downloaded from a remote location via the interface and communications channel from the Internet or another network location or site. The computer system includes the computer readable medium having such software or program code recorded such that instructions of the software or the program code can be carried out. The use of the computer system preferably affects advantageous apparatuses for constructing a runtime symbol table for a computer program in accordance with the embodiments of the invention.

The following overview of is an example of insulin administered at various points of times and insulin prescribed in a treatment regiment for the diabetic patient, respectively and their current sums and difference between these sums can be displayed on the display of said device indicated by means of the FIGS. 3, 4, 5, 6 and 7.

TABLE 1

| Point of time | 06:00 | 06:30 | 12:00 | 18:00 | 19:00 |
|---|---|---|---|---|---|
| Insulin administered | 4 | 3 | 6 | 5 | 1 |
| Current Sum of insulin administered | 4 | 7 | 13 | 18 | 19 |
| Insulin prescribed | 8 | | 7 | 5 | |
| Current Sum of insulin prescribed | 8 | 8 | 15 | 20 | 20 |
| Difference | −4 | −1 | −2 | −2 | −1 |

FIG. 3 shows a display showing provided logged doses of insulin administered. However the same data could also be presented by a display of the line “insulin administered” from table 1. Said provided logged insulin doses administered may be keyed in or dialed on said device, or transferred from an external device. Accordingly, parts of data, i.e. on or more insulin administrations, or all data can for a registration period be displayed. Further more it is possible to display the points of times where administrations took place. Hereby, the diabetic patient can be informed about doses of insulin administered after the actual administration and when it was administered.

FIG. 4 shows a display showing provided currently summed logged doses of insulin administered. However the same data could also be presented by a display of the line “current sum of insulin administered” from table 1. Said provided logged insulin doses administered may be keyed in or dialed on said device prior to summation. Accordingly, parts of summed logged doses or all of said summed logged doses can for a registration period be displayed. Further more it is possible to display the points of times of administrations relating to said sums, i.e. at what time where the summed administrations performed. As an example—se FIG. 4—the value 7 is a sum of 4 and 3 for these two insulin administrations, this corresponds to the sum of 7 at 6:30 in table 1. Hereby, the diabetic patient can be informed about current summed doses of insulin after the actual administration and when corresponding doses actually were administered.

FIG. 5 shows a display showing dose of insulin to be administered in a treatment regimen. However the same data could also be presented by a display of the line “insulin prescribed” from table 1. Said insulin doses to be administered may be keyed in or dialed on said device. Accordingly, parts of data or all data—in both cases insulin to be administered at various points of times can for a registration period be displayed. Furthermore it is possible to display the points of times of prescribed administrations. Hereby, the diabetic patient can be informed about doses of insulin to be administered in a treatment regimen prior to actual administration and when to administer.

FIG. 6 shows a display showing currently summed dose of insulin to be administered in a treatment regimen. However the same data could also be presented by a display of the line “current sum of insulin prescribed” from table 1. Said insulin doses to be administered may be keyed in or dialed on said device.

Accordingly, parts of doses of insulin to be administered or all of said doses of insulin to be administered can for a registration period be displayed prior to any actual administration. Further more it is possible to display the points of planned times of administrations relating to said sums. As an example—se FIG. 6—the value 15 is a sum of 8 and 7 for these prescribed two insulin administrations, this corresponds to the sum of 15 at 12:00 in table 1. Hereby, the diabetic patient can be informed about summed doses of insulin to be administered in a treatment regimen when, i.e. point of times e.g. 06:00, 12:00, etc, these sums should be achieved.

The display of said data may be implemented on the device as follows:

The processor may be configured to receive (keyed in, from a remote device, etc) data comprising doses of bolus insulin administration, e.g. as prescribed for the diabetic patient by his physician for a certain period, e.g. a registration period. By means of the data the processor can then calculate a number of current sums of doses of bolus insulin, and accordingly the processor could then cause the display to display one or more of said number of current sums.

Furthermore, the processor may cause the display to display one or more points of times of prescribed administrations relating to said number of current sums, e.g. at time of meals, i.e. 06:00, 12:00, 18:00.

FIGS. 7 and 8 show a display showing differences between insulin administered and prescribed at points of times during a registration period. FIG. 7 relates to the same data which could also be presented by a display of the line “difference” from table 1, whereas FIG. 8 relates to the same data which could also be presented by a display of the line “difference” from table 2 below.

The following overview of insulin administered at various points of times and insulin prescribed in a treatment regiment for the diabetic patient, respectively and their current sums. Difference between these sums is indicated by means of FIG. 8.

TABLE 2

| | Point of time | | | | |
|---|---|---|---|---|---|
| | 06:00 | 06:30 | 12:00 | 18:00 | 19:00 |
| Insulin administrered | 4 | 5 | 6 | 7 | 1 |
| Current Sum of insulin administered | 4 | 9 | 15 | 22 | 23 |
| | Point of time | | | | |
| | 06:00 | 06:30 | 12:00 | 18:00 | 19:00 |
| Insulin prescribed | 8 | | 7 | 5 | |
| Current Sum of insulin prescribed | 8 | 8 | 15 | 20 | 20 |
| | Point of time in the | | | | |
| | registration period | | | | |
| | 06:00 | 06:30 | 12:00 | 18:00 | 19:00 |
| Difference | −4 | 1 | 0 | 2 | 3 |

Hereby a diabetic patient can be informed about doses of insulin administered in relation to the treatment regimen, i.e. insulin prescribed for the diabetic patient for a certain period, e.g. the registration period as discussed.

The display of said data may be implemented on the device as follows:
The processor of the device may be provided with data comprising logged doses of bolus insulin administrations in a registration period, corresponding to data in table 2, i.e. 4, 5, 6, 7 and 1.
Further more, the processor of the device can also be provided with data comprising doses of bolus insulin administrations prescribed for the diabetic patient for the same registration period, corresponding to data in table 2, i.e. 8, 7 and 5; typically these data is the treatment regimen for the patient.

Accordingly, the processor can then calculate a first number of current sums from said provided logged doses of bolus insulin administrations in said registration period, i.e. 4, 9, 15, 22 and 23 IU.

Accordingly, the processor can then also calculate a second number of current sums from doses of bolus insulin administrations prescribed for the diabetic patient in said registration period, i.e. 8, 8, 15, 20 and 20 International Units.

Consequently, the processor in the device can then calculate a number of differences between said first and second number of current sums, i.e. a number of differences between “4, 9, 15, 22 and 23” and “8, 8, 15, 20 and 20” which give −4, −1, 0, 2 and 3. Any positive difference could indicate more insulin administered than prescribed at the point of time of administration in the registration period, conversely, any negative difference could then indicate less insulin administered than prescribed at the point of time of administration in the registration period passed.

Subsequently, the processor can the cause the display to display one or more of said number of differences as shown in FIG. 8 and/or as shown in table 2.

Thus the displayed graph shows running differences between summed actual medications and prescribed medication from said registration period, which preferably is a day.

Furthermore, the processor can the cause the display to display point of times of said number of differences—which is point of times of actual bolus insulin administrations for the registration period passed—as shown in FIG. 8, i.e. at 06:00, 6:30, 12:00, 18:00 and 19:00 hours.

FIG. 9 shows a display showing sums of insulin administered during eight registration periods. Alternatively or additionally, data could be displayed as shown in table 3 below.

TABLE 3

| | |
|---|---|
| Sum of insulin administered, reg. period 1 | 19 |
| Sum of insulin administered, reg. period 2 | 17 |
| Sum of insulin administered, reg. period 3 | 16 |
| Sum of insulin administered, reg. period 4 | 17 |
| Sum of insulin administered, reg. period 5 | 18 |
| Sum of insulin administered, reg. period 6 | 19 |
| Sum of insulin administered, reg. period 7 | 20 |
| Sum of insulin administered, reg. period 8 | 22 |

Thus the diabetic patient can hereby be informed about doses of insulin administered in a period of time passed. Typically said period of time passed is two or more registration periods of equal length. More registration periods could comprise a week, a month, a quarter or any other period determined prior to display by the diabetic patient. By application of the same length for each registration period, it makes more sense to compare results between periods, further more when data are displayed in a bar graph, pie chart, as a line in a x-y diagram any trend, e.g. an increasing trend, a decreasing trend or status quo for insulin administered can easily be spotted by a quick glance.

The display of doses of insulin administered in a period of time passed, i.e. for more registration periods typically of equal length, may be implemented on the device as follows:

The processor could compute a sum of logged doses of bolus insulin administered for each registration period, e.g. 19 for the sum of the first period as shown in table 1. Likewise, data for the other seven periods could be computed with the result as show in table 3 above.

Subsequently, the processor as embedded in the device could cause the display to display said sum for sum of logged doses of bolus insulin administered for each registration period, e.g. for two, five or eight registration periods as shown in the figure.

Furthermore, the processor could then cause the display to display data identifying each of said registration periods, such as date, day number, week number, quarter, year.

By a quick glance on FIG. 9, an increasing trend for insulin administered can easily be spotted. The patient could wonder why increasingly more bolus insulin are administered—is it because more was prescribed—or is he or she having a high consumption of food and therefore needs more meal related insulin, i.e. bolus insulin. The patient could thereby predict an indirect weight increase since—while having a high (and higher) consumption of food—he or she most likely will gain weight, which finally could cause obesity problems.

FIGS. 10 and 11 show a display showing differences between insulin administered and prescribed from eight registration periods. Data as indicated in FIG. 10 could instead be displayed as the column “difference” in table 4 below.

TABLE 4

| | Sum of | | | |
|---|---|---|---|---|
| | Administ | Prescribed | Difference | differences |
| Sum of insulin administered reg. period 1 | 19 | 20 | −1 | −1 |
| Sum of insulin administered reg. period 2 | 17 | 20 | −3 | −4 |
| Sum of insulin administered reg. period 3 | 16 | 20 | −4 | −8 |
| Sum of insulin administered reg. period 4 | 17 | 20 | −3 | −11 |
| Sum of insulin administered reg. period 5 | 18 | 20 | −2 | −13 |
| Sum of insulin administered reg. period 6 | 19 | 20 | −1 | −14 |
| Sum of insulin administered reg. period 7 | 20 | 20 | 0 | −14 |
| Sum of insulin administered reg. period 8 | 22 | 20 | 2 | −12 |

As another example:

TABLE 5

| | Sum of | | | |
|---|---|---|---|---|
| | Administ | Prescribed | Difference | differences |
| Sum of insulin administered reg. period 1 | 19 | 18 | 1 | −1 |
| Sum of insulin administered reg. period 2 | 20 | 18 | 2 | 1 |
| Sum of insulin administered reg. period 3 | 16 | 18 | −2 | −1 |
| Sum of insulin administered reg. period 4 | 20 | 18 | 2 | 1 |
| Sum of insulin administered reg. period 5 | 18 | 18 | 0 | 1 |
| Sum of insulin administered reg. period 6 | 19 | 18 | 1 | 2 |
| Sum of insulin administered reg. period 7 | 20 | 18 | 2 | 4 |
| Sum of insulin administered reg. period 8 | 22 | 18 | 4 | 8 |

Data as indicated in FIG. 11 could instead be displayed as the column “difference” as in table 5 above.

The display of differences between insulin administered and prescribed from any number of registration periods may be implemented on the device as follows:

The processor in the device should calculate a number of differences between:

a computed sum of logged doses of bolus insulin administered for each registration period, e.g. from table 5, i.e. 19 IU, 20 IU, 16 IU, 20 IU, 18 IU, 19 IU, 20 IU and 22 IU, and
a computed sum of prescribed doses of bolus insulin to be administered for each registration period, e.g. from table 5, i.e. 18 IU for all registration periods.

These differences will e.g. from table 5 be as follows 1 IU, 2 IU, −2 IU, 2 IU, 0 IU, 1 IU, 2 IU and 4 IU, respectively.

The processor should the cause the display to display two or more number of differences for corresponding registration periods, i.e. 1 IU, 2 IU, −2 IU, 2 IU, 0 IU, 1 IU, 2 IU and 4 IU as shown in FIG. 11, a display format as shown in table 5 could be applied as well.

In the display a positive difference indicates more insulin administered than prescribed for the corresponding registration period, and, conversely any negative difference indicates less insulin administered than prescribed for the corresponding registration period.

Furthermore, the processor could then cause the display to display data identifying each ofsaid registration periods, such as date, day number, week number, quarter, year.

FIG. 10 corresponding to table 4 is an example of less insulin administered than prescribed for most of the registration periods, i.e. only more was administered than prescribed in period #8, administered as prescribed for period #7, whereas for period #'s 1 to 6 and 8 less doses of bolus insulin were administered than prescribed, namely 1 IU, 3 IU, 4 IU, 3 IU, 2 IU and 1 IU, respectively.

Conversely, FIG. 11 corresponding to table 5 is an example of more insulin administered than prescribed for most of the time passed, i.e. said registration periods. As can be seen—when comparing to table 4: 2 IUs of insulin are prescribed less, namely 18 IU instead of 20 IU. Thus, less bolus insulin was only administered than prescribed in period #3, bolus insulin was administered as prescribed for period #5, however for period #'s 1, 2, 4 and 6, 7 and 9 more doses of bolus insulin were administered than prescribed, namely 1 IU, 2 IU, 2 IU, 1 IU, 2 IU and 4 IU, respectively.

By the application of the same length for each registration period, it makes more sense to compare results, i.e. differences between insulin administered and prescribed, between periods. Further more when data are displayed in a bar graph, pie chart, as a line in a x-y diagram, any trend, e.g. an increasing trend, a decreasing trend or status quo for insulin administered in relation to what actually were prescribed can easily be spotted by a quick glance.

FIGS. 12 and 13 show a display showing the summed differences from FIGS. 10 and 11 for eight registration periods. Said eight registration periods are just an exemplary embodiment.

Data relating to FIG. 10 (table 4) forms the basis for the data shown in FIG. 12, likewise data relating to FIG. 11 (table 5) forms the basis for the data shown in FIG. 13.

In the example from table 4, the sums of differences are mostly a number of decreasing numbers, i.e. −1 IU, −4 IU, −8 IU, −11 IU, −13 IU, −14 IU and −12 IU (see also FIG. 12), thus there is for the last periods a decreasing trend, thus by a quick glance on FIG. 12, a decreasing trend for less (i.e. a smaller dosage) bolus insulin administered than prescribed can easily be spotted.

Conversely, in the example from table 5, the sums of differences are primarily (except for the first five registrations periods “1”, “2”, “3”, “4” and “5”, where the trend is fluctuating up and down) increasing numbers, i.e. 2 IU, 4 IU and 8 IU,—see also FIG. 13—thus there is for the last periods an increasing trend, which could be interpreted to mean an increasing trend for more (i.e. a higher dosage) bolus insulin administered than prescribed. If this continues, still more bolus insulin is administered or just on a higher level than prescribed, the latter is 18 IU, e.g. 19 IU, 20 IU or even more bolus insulin is administered as a total for each registration period, e.g. a day, this could cause obesity problem or at least a weight gain over time. The cause could be that he or she actually had a high consumption of food in the registration periods just passed (the period of time passed) and therefore at these past occasions felt a need for more meal related insulin, i.e. bolus insulin e.g. 19 IU, 20 IU which were more than the prescribed dose (18 IU) for the entire day. The patient could thereby foresee an indirect weight increase, since—while consuming more food—he or she most likely will gain weight, which finally could cause obesity problems. Maybe his scale will not tell, but data as displayed in FIG. 13 could indicate a potential weight gain.

The processor in the device should calculate a number of differences between:

a computed sum of logged doses of bolus insulin administered for each registration period, e.g. from table 5, i.e. 19 IU, 20 IU, 16 IU, 20 IU, 18 IU, 19 IU, 20 IU and 22 IU, and
a computed sum of prescribed doses of bolus insulin to be administered for each registration period, e.g. from table 5, i.e. 18 IU for all registration periods.

The processor should further calculate a number of currents sums based on said number of differences for a corresponding number of registration periods, i.e. −1 IU, 1 IU, −1 IU, 1 IU, 1 IU, 2 IU, 4 IU and 8 IU as the “sum of differences” in table 5.

Consequently, the processor should cause the display to display one or more of said number of currents sums for corresponding registration periods, which in an exemplary embodiment is 8 days.

If the “sum of differences” in table 5, especially the last three items, i.e. 2 IU, 4 IU and 8 IU are shown in a graphical way, it is easy to spot an increasing trend indicating more bolus insulin administered than prescribed.

Any positive displayed current sum indicates more insulin administered than prescribed, and where any negative displayed current sum indicates less insulin administered than prescribed.

Further more, the processor could cause the display to display data days as dates or day numbers identifying said registration periods in the example given.

In all embodiments, it may be the case that said provided logged insulin doses administered can be received from an external device prior to administration.

Further, in all embodiments, it may also be the case that said provided doses of bolus insulin in said treatment regimen, i.e. insulin doses to be administered, are received from an external device prior to the display of these values in various forms.

Correspondingly, said provided logged insulin doses administered or to be administered and may be keyed in or dialed on said device. Furthermore, insulin in the treatment regimen and doses actually administrated may be received from an external device.

The registration period can be one of:
an initial setting a number of awake hours for the diabetic patient,
one or more days,
a number of hours,
a number of hours and minutes.
Examples are a specific date, a day number, a week number, a quarter and year or more of said examples.

Another number of registration periods could be applied, e.g. seven where a single registration period could be chosen to be a day, thus giving a week as the entire period analyzed. Correspondingly, another number of registration periods could be applied, e.g. 365 where the single registration period could be chosen to be a day, thereby giving a year as the entire period analyzed. Likewise, another number of registration periods could be applied, e.g. 4 registration periods, where the single registration period could be chosen to be a week, thereby giving a month as the entire period analyzed. Likewise, another number of registration periods could be applied, e.g. 30 days, i.e. where the single registration period is the day, thus again obtaining the month comprising 30 days as the entire period analyzed. Registration periods typically would be chosen as a period of time passed as the entire period of interest for a subsequent analysis of data.

Said registration period preferably is a day.

Said registration period can be determined by the diabetic patient, or be an initial setting on the device. Correspondingly, the diabetic patient may determine which number of registration period, which registration periods to display and which parts, e.g. certain point of times, of which registration period to display.

Said device may be a drug administration device, preferably a syringe or a blood glucose measuring device.

Some preferred embodiments have been shown in the foregoing, but it should be stressed that the invention is not limited to these, but may be embodied in other ways within the subject matter defined in the following claims.

Any combination of the above-described elements in all possible variations thereof is encompassed by the invention unless otherwise indicated herein or otherwise clearly contradicted by context.

The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects presented herein to the maximum extent permitted by applicable law.
All headings and sub-headings are used herein for convenience only and should not be construed as limiting the invention in any way.
The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.
The citation and incorporation of patent documents herein is done for convenience only and does not reflect any view of the validity, patentability, and/or enforceability of such patent documents.
This invention includes all modifications and equivalents of the subject matter recited in the claims appended hereto as permitted by applicable law.

1. A device (1) for informing a diabetic patient about dose of insulin administered, said device comprising:
a display (8) for displaying graphics, text and/or combinations thereof,
a processor (230) that is interfaced with said display,
wherein the processor is configured to:
be provided with data comprising logged doses of bolus insulin administrations in a registration period, and
cause the display to display two or more of said provided logged doses.

2. A device (1) according to claim 1 wherein said data further comprises the points of times for administration in said registration period, and wherein the processor is further configured to:
cause the display to display two or more corresponding point of times of administrations.

3. A device (1) for informing a diabetic patient about dose of insulin administered, said device comprising:
a display (8) for displaying graphics, text and/or combinations thereof,
a processor (230) that is interfaced with said display,
wherein the processor is configured to:
be provided with data comprising logged doses of bolus insulin administrations in a registration period, and
calculate a number of current sums of said provided logged doses of bolus insulin administrations, and
cause the display to display two or more of said number of current sums of said provided logged doses of bolus insulin administrations.

4. A device (1) according to claim 3, where said data further comprises the points of times of administration in said registration period, and wherein the processor is further configured to:
cause the display to display two or more corresponding points of times of administration.

5. A device (1) for informing a diabetic patient about dose of insulin to be administered in a treatment regimen, said device comprising:
a display (8) for displaying graphics, text and/or combinations thereof,
a processor (230) that is interfaced with said display,
wherein the processor is configured to:
be provided with data comprising doses of bolus insulin administration prescribed for the diabetic patient in a registration period, and
cause the display to display one or more of said doses.

6. A device (1) according to claim 5, wherein said data further comprises the point of times at which said bolus insulin is to be administered in said registration period and wherein the processor is further configured to:
cause the display to display one or more points of times of prescribed administration.

7. A device (1) for informing a diabetic patient about dose of insulin to be administered in a treatment regimen, said device comprising:
a display (8) for displaying graphics, text and/or combinations thereof,
a processor (230) that is interfaced with said display,
wherein the processor is configured to:
be provided with data comprising doses of bolus insulin administration prescribed for the diabetic patient in a registration period,
calculate a number of current sums of doses of bolus insulin from said treatment regimen in said registration period, and
cause the display to display one or more of said number of current sums.

8. A device (1) according to claim 7, wherein said data further comprises the point of times at which said bolus insulin is to be administered in said registration period and wherein the processor is further configured to:
cause the display to display one or more points of times of prescribed administration relating to said number of current sums.

9. A device (1) for informing a diabetic patient about doses of insulin administered in relation to the treatment regimen for the diabetic patient, said device comprising:
a display (8) for displaying graphics, text and/or combinations thereof,
a processor (230) that is interfaced with said display,
wherein the processor is configured to:
1) be provided with data comprising logged doses of bolus insulin administrations in a registration period,
2) be provided with data comprising doses of bolus insulin administrations prescribed for the diabetic patient in said registration period,
calculate a first number of current sums from 1) said provided logged doses of bolus insulin administrations in said registration period,
calculate a second number of current sums from 2) doses of bolus insulin administrations prescribed for the diabetic patient in said registration period,
calculate a number of differences between said first and second number of current sums, where any positive difference indicates more bolus insulin administered than prescribed at the point of time of administration in the registration period, and where any negative difference indicates less bolus insulin administered than prescribed at the point of time of administration in the registration period, and
cause the display to display one or more of said number of differences.

10. A device (1) according to claim 9, wherein said data comprising logged doses of bolus insulin administrations in the registration period further comprising the points of times of administration in said registration period, and wherein the processor is further configured to:
cause the display to display one or more corresponding points of times of administration.

11. A device (1) for informing a diabetic patient about doses of insulin administered in a period of time passed, said device comprising:
a display (8) for displaying graphics, text and/or combinations thereof,
a processor (230) that is interfaced with said display,
wherein the processor is configured to:
from said period of time passed, which is two or more registration periods of equal length,
compute a sum of logged doses of bolus insulin administered for each registration period, and
cause the display to display said sum for two or more corresponding registration periods.

12. A device (1) according to claim 11, wherein the processor is further configured to cause the display to display data, such as date, day number, week number, quarter, year identifying each of said registration periods.

13. A device (1) for informing a diabetic patient about doses of insulin administered in relation to a treatment regimen for the diabetic patient in a period of time passed, said device comprising:
a display (8) for displaying graphics, text and/or combinations thereof,
a processor (230) that is interfaced with said display,
wherein the processor is configured to:
from said period of time passed, which is two or more registration periods of equal length, calculate a number of differences between:
a computed sum of logged doses of bolus insulin administered for each registration period,
a computed sum of prescribed doses of bolus insulin to be administered for each registration period,
wherein the processor is further configured to:
cause the display to display two or more number of differences for corresponding registration periods, where a positive difference indicates more bolus insulin administered than prescribed for the corresponding registration period, and where a negative difference indicates less bolus insulin administered than prescribed for the corresponding registration period.

14. A device (1) according to claim 13, wherein the processor is further configured to cause the display to display data, such as date, day number, week number, quarter, year identifying each of said registration periods.

15. A device (1) for informing a diabetic patient about doses of insulin administered in relation to the treatment regimen for the diabetic patient in a period of time passed, said device comprising:
a display (8) for displaying graphics, text and/or combinations thereof,
a processor (230) that is interfaced with said display,
wherein the processor is configured to:
from said period of time passed, which is two or more registration periods of equal length, calculate a number of differences between:
a computed sum of logged doses of bolus insulin administered for each registration period,
a computed sum of prescribed doses of bolus insulin to be administered for each registration period,
wherein the processor is further configured to:
calculate a number of currents sums based on said number of differences for a corresponding number of registration periods, and
cause the display to display one or more of said number of currents sums for corresponding registration periods, whereby any positive displayed current sum indicates more bolus insulin administered than prescribed, and where any negative displayed current sum indicates less bolus insulin administered than prescribed.

16. A device (1) according to claim 15, wherein the processor is further configured to cause the display to display data, such as date, day number, week number, quarter, year identifying each of said registration periods.

17. A device (1) according to claim 1, wherein said provided logged bolus insulin doses administered and/or said provided doses of bolus insulin in said treatment regimen for the diabetic patient are received from an external device.

18. A device (1) according to claim 1, wherein said provided logged bolus insulin doses administered and/or said provided doses of bolus insulin in said treatment regimen for the diabetic patient are keyed in, dialed on said device or is received from another device.

19. A device (1) according to claim 2, wherein said registration period is selected from the group of:
an initial setting a number of awake hours for the diabetic patient,
one or more days,
a number of hours,
a number of hours and minutes.

20. A device (1) according to claim 1, wherein said registration period is a day.

21. A device (1) according to claim 2, wherein said device is a drug administration device, preferably a syringe.
